(19) United States
(12) Patent Application Publication
(10) Pub. No.: US 2021/0220523 A1
(43) Pub. Date: Jul. 22, 2021

(54) NO RELEASING COATED PROSTHETIC VASCULAR GRAFTS

(71) Applicants: Ronald J. SHEBUSKI, Melbourne, FL (US); University of Georgia Research Foundation, Inc., Athens, GA (US)

(72) Inventors: Ronald J. SHEBUSKI, Melbourne, FL (US); Hitesh HANDA, Athens, GA (US); Sean HOPKINS, Athens, GA (US)

(21) Appl. No.: 17/270,675

(22) PCT Filed: Aug. 22, 2019
(86) PCT No.: PCT/US2019/047749

§ 371 Date: Feb. 23, 2021

Related U.S. Application Data
(60) Provisional application No. 62/722,533, filed on Aug. 24, 2018.

(51) Int. Cl. A61L 27/54; A61K 33/00; A61L 27/44; A61L 27/50; A61L 27/16; A61L 27/18; A61L 27/04; A61K 47/59; A61L 27/34; A61K 47/69

(52) U.S. Cl. CPC A61L 27/54; A61K 33/00; A61L 27/446; A61L 27/507; A61L 27/16; A61L 27/18; A61L 2300/606; A61K 47/59; A61L 27/34; A61K 47/6957; A61L 2300/114; A61L 2300/44; A61L 2420/06; A61L 27/04

(57) ABSTRACT

A variety of nitric oxide-releasing vascular grafts and prostheses are provided. Methods of making the nitric oxide-releasing vascular grafts and prostheses are also provided. Methods of administering the nitric oxide-releasing vascular grafts and prostheses to a subject in need thereof are also provided. The nitric oxide-releasing vascular grafts and prostheses have a base layer made of a graft material and a nitric oxide-releasing layer made from a polymer matrix including a plurality of polysiloxanes and a plurality of nitric oxide-donating crosslinking moieties covalently crosslinking polysiloxanes in the plurality of polysiloxanes. In some aspects, the vascular grafts and prostheses can provide for reduced infection rates and increased patency by providing for prolonged local delivery of nitric oxide when implanted in a vessel of a subject in need thereof.

### CROSS-REFERENCE TO RELATED APPLICATIONS

This application claims the benefit of and priority to U.S. Provisional Application Ser. No. 62/722,533, having the title “NO RELEASING COATED PROSTHETIC VASCULAR GRAFTS”, filed on Aug. 24, 2018, the disclosure of which is incorporated herein by reference in its entirety.

### BACKGROUND

Local drug delivery to sites of disease or injury in the body remains a daunting problem. While efficacious drugs have been identified and characterized for many diseases, controlled delivery of such drugs at a sufficient concentration for a sufficient amount of time while avoiding detrimental systemic side effects remains elusive. Local delivery strategies are intended to maximize the drug effect at the target site and minimize off target drug effects. Blood carrying conduits that are surgically placed into patients for various conditions to be described below, represent a highly reasonable site for local drug delivery as these conduits unfortunately fail with remarkable consistency.

Blood carrying conduits may be constructed of either native vein or prosthetic materials such as expanded polytetrafluoroethylene (ePTFE) or polyurethane. Two common indications for the use of prosthetic materials are: 1) arterio-venous (A-V) access in which needle insertion sites enable blood access for hemodialysis that is performed 2-3 times per week in end stage renal disease (ESRD) patients and 2) peripheral vascular disease (PVD) which is the result of atherosclerosis causing arterial obstruction with pain and cramping in the legs. A blood conduit, either prosthetic or native vein, is often used to bypass the obstructed artery. The durability and long-term patency of blood conduits used to replace diseased arteries in PVD are substantially better than results with grafts used to provide chronic blood access for hemodialysis.

More than 75,000 new hemodialysis grafts are placed in the U.S. each year and costs for creating and maintaining these grafts exceed $1 billion annually. Over 80% of ePTFE arteriovenous access grafts and 20% of peripheral arterial bypass grafts will fail or become dysfunctional each year resulting in considerable patient morbidity and substantial costs to the healthcare system. Graft failure is often due to neointimal hyperplasia caused by mechanical injury (e.g., high blood flow) to the venous outflow tract of the conduit.

Various delivery methods have been utilized using primarily two antiproliferative drugs in cardiovascular coronary applications: sirolimus (and analogs) and paclitaxel. Antiproliferative drugs have gained considerable popularity and use in the cardiovascular arena with the realization that neointimal hyperplasia could be interrupted by these agents, albeit by disparate mechanisms to be described below. Neointimal hyperplasia refers to the accumulation of rapidly proliferating smooth muscle cells and fibroblasts that eventually form an obstructive lesion. Following routine angioplasty procedures, obstructive restenotic lesions diminish blood flow over time and antiproliferative drugs applied locally prevent such lesions to a large degree.

Sirolimus, also known as rapamycin, is a cytostatic (Gi to S cell cycle interruption) compound used to coat drug-eluting coronary stents (Cypher®), prevent organ transplant rejection (Rapamune®) and to treat a rare lung disease called lymphangioleiomyomatosis. It has immunosuppressant functions in humans and is especially useful in preventing the rejection of kidney transplants. Sirolimus inhibits activation of T cells and B cells by reducing the production of interleukin-2 (IL-2). Other mTOR inhibitory analogs that have a similar mechanism of action to sirolimus are everolimus, zotarolimus, tacrolimus, pimecrolimus, temsirolimus, ridaforolimus and biolimus. In fact, newer generation coronary drug-eluting stents elute either sirolimus (Orsiro®), everolimus (Xience®) or zotarolimus (Resolute Integrity®).

The other antiproliferative agent utilized in the cardiovascular arena to suppress intimal hyperplasia is paclitaxel. Unlike cytostatic sirolimus, paclitaxel is cytotoxic and has been used for decades as an anti-cancer agent. The only paclitaxel drug-eluting coronary stent was Taxus® which is now off the market as well. No other paclitaxel-eluting drug coated stents have been developed to date. However, paclitaxel is the agent of choice for drug eluting balloons, which have just entered the marketplace recently (Lutonix®, Bard).

There remains a need for improved vascular grafts that overcome the aforementioned deficiencies.

### SUMMARY

Embodiments of the present disclosure provide for implantable vascular grafts, methods of making vascular grafts, methods of use, and the like.

An embodiment of the present disclosure includes an implantable vascular graft which includes a tubular base layer including a graft material. The tubular base defines a luminal surface and an abluminal surface. The implantable vascular graft also includes a nitric oxide-releasing layer, which can be disposed on one or both of the luminal or the abluminal surfaces. The nitric oxide-releasing layer can include a polymer matrix. The polymer matrix can include (i) a plurality of polysiloxanes; and (ii) a plurality of nitric oxide-donating crosslinking moieties that covalently crosslink polysiloxanes in the plurality of polysiloxanes.

An embodiment of the present disclosure also includes methods of making an implantable vascular graft. Another embodiment includes methods of administering a vascular graft to an endoluminal surface of a vessel of a subject in need thereof. The method can include intraluminally inserting a vascular graft as described above and positioning the vascular graft at a location in the vessel via a positioning apparatus. The vascular graft cab be expanded and anchored at a location in the vessel of the subject.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 is an example of a possible embodiment of a NO releasing graft material according to the present disclosure.

FIG. 2 is a bar graph of the measured NO flux (×10-10 mol cm-2 min-1) from the luminal surface as a function of the number of days under physiological conditions for SNAP-PDMS ePTFE vascular grafts and for SNAP-PDMS ePTFE vascular grafts where the luminal surface has been precoated with polydopamine for either 24 hours or 48 hours.

### DETAILED DESCRIPTION

In various aspects, vascular prosthesis and graft materials are provided having at least one surface coated with a nitric oxide donor material. The materials and devices can function as nitric oxide-releasing materials to provide local delivery of nitric oxide when in use. In an exemplary aspect, S-nitroso-N-acetylpenicillamine (SNAP) is used as a nitric oxide-donor material covalently attached to polydimethylsiloxane (PDMS), in the form of a thin polymeric topcoat on a vascular graft or prosthesis. The SNAP-PDMS coated vascular grafts can, in some aspects, prevent one or more of cell growth, neointimal hyperplasia, thrombus formation and bacterial adhesion in prosthetic AV access grafts and prosthetic peripheral vascular grafts.

NOREL (Nitric Oxide Releasing Agents), also known as NO donors or carriers, release exogenous Nitric Oxide (NO) that has profound and potent pharmacological actions. Specifically, released NO has been demonstrated to exert both potent cellular anti-proliferative (Napoli et al., 2013) activity and anti-microbial activity (Scheirer et al., 2012). Prosthetic graft failure is often due to neointimal hyperplasia caused by mechanical injury (e.g., high blood flow) to the venous outflow tract of the AV access conduit. While drugs that inhibit neointimal hyperplasia are available, delivery of these drugs to the site of injury at a sufficient dose for a sufficient and prolonged period of time has been challenging. In some aspects, the nitric oxide-releasing vascular grafts and prosthesis described herein can provide for prolonged, local delivery of nitric oxide.

Conventional prosthetic vascular grafts often fail for one or both of two reasons. One reason for failure is neointimal hyperplasia that is induced by proliferation of smooth muscle cells that have accumulated at the venous outflow tract of the AV access conduit and eventually diminish blood flow through the conduit. The other reason that prosthetic vascular grafts fail is due to infections. The nitric oxide-releasing vascular grafts and prosthesis described herein can, in some aspects, address both cell proliferation and infection, inhibiting both processes through disparate mechanisms.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting. The skilled artisan will recognize many variants and adaptations of the embodiments described herein. These variants and adaptations are intended to be included in the teachings of this disclosure and to be encompassed by the claims herein.

Although any methods and materials similar or equivalent to those described herein can also be used in the practice or testing of the present disclosure, the preferred methods and materials are now described. Functions or constructions well-known in the art may not be described in detail for brevity and/or clarity. Embodiments of the present disclosure will employ, unless otherwise indicated, techniques of nanotechnology, organic chemistry, material science and engineering and the like, which are within the skill of the art. Such techniques are explained fully in the literature.

It should be noted that ratios, concentrations, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In some embodiments, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

### Definitions

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used.

As used herein, “endolumenally,” “intraluminally” or “transluminal” all refer synonymously to implantation placement by procedures wherein the prosthesis is advanced within and through the lumen of a body vessel from a remote location to a target site within the body vessel. In vascular procedures, a medical device will typically be introduced “endovascularly” using a catheter over a wire guide under fluoroscopic guidance. The catheters and wire guides may be introduced through conventional access sites to the vascular system.

As used herein, the terms “vessel” or “body vessel” mean any body passage lumen that conducts fluid, including but not limited to blood vessels, esophageal, intestinal, biliary, urethral and ureteral passages. The vessels can include a vein, an artery, a biliary duct, a ureteral vessel, a portion of the alimentary canal, and other bodily vessels.

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel.

Unless otherwise indicated, as used herein, a “layer” refers to a portion of a structure having a defined composition or structure and a defined boundary with respect to an adjacent material. A layer of a material may be deposited by deposition (e.g. spray deposition) of a polymer solution in multiple deposition events. For example, a single layer may be formed by deposition of material in separate portions, where no definite boundary of structure or composition is present between the material deposited in the first and subsequent portions. Furthermore, a single layer may be formed by spray deposition of a first portion of a deposited material followed by drying of the deposited material and subsequent spray deposition of a second portion of material with the same composition onto the dried deposited material, provided that the deposited material does not include a structural or compositional boundary between the first deposited material and the second deposited material.

The term “luminal surface” or “luminal side,” as used herein, refers to the portion of the surface area of a medical device defining at least a portion of an interior lumen. Conversely, the term “abluminal surface” or “abluminal side,” as used herein, refers to portions of the surface area of a medical device that do not define at least a portion of an interior lumen. For example, where the medical device is a tubular frame defining a cylindrical lumen, the abluminal surface can include the exterior surface, sides and edges of the tubular frame, while the luminal surface can include the interior surface of the tubular frame.

The term “mixture” refers to a combination of two or more substances in which each substance retains its own chemical identity and properties.

The terms “frame” and “support frame” are used interchangeably herein to refer to a structure that can be implanted, or adapted for implantation, within the lumen of a body vessel.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In some embodiments, a straight chain or branched chain alkyl has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g. have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons (e.g., from one to six carbon atoms) in its backbone structure. Likewise, “lower alkenyl” and “lower alkynyl” have similar chain lengths. Throughout the application, preferred alkyl groups are lower alkyls. In some embodiments, a substituent designated herein as alkyl is a lower alkyl.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl”, as used herein, refers to straight or branched chain, or cyclic carbon-containing radicals, or combinations thereof, containing at least one heteroatom. Suitable heteroatoms include, but are not limited to, 0, N, Si, P, Se, B, and S, wherein the phosphorous and sulfur atoms are optionally oxidized, and the nitrogen heteroatom is optionally quaternized. Heteroalkyls can be substituted as defined above for alkyl groups.

The term “alkylthio” refers to an alkyl group, as defined above, having a sulfur radical attached thereto. In some embodiments, the “alkylthio” moiety is represented by one of —S-alkyl, —S-alkenyl, and —S-alkynyl. Representative alkylthio groups include methylthio, and ethylthio. The term “alkylthio” also encompasses cycloalkyl groups, alkene and cycloalkene groups, and alkyne groups. “Arylthio” refers to aryl or heteroaryl groups. Alkylthio groups can be substituted as defined above for alkyl groups.

The terms “alkenyl” and “alkynyl”, refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, and tert-butoxy. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The terms “amine” and “amino” are art-recognized and refer to both unsubstituted and substituted amines, e.g., a moiety that can be represented by the general formula:

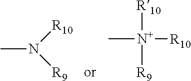

wherein R₉, R₁₀, and R′₁₀ each independently represent a hydrogen, an alkyl, an alkenyl, —(CH₂)ₘ—R₈ or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In still other embodiments, the term “amine” does not encompass amides, e.g., wherein one of R₉ and R₁₀ represents a carbonyl. In additional embodiments, R₉ and R₁₀ (and optionally R′₁₀) each independently represent a hydrogen, an alkyl or cycloalkyl, an alkenyl or cycloalkenyl, or alkynyl. Thus, the term “alkylamine” as used herein means an amine group, as defined above, having a substituted (as described above for alkyl) or unsubstituted alkyl attached thereto, e.g., at least one of R₉ and R₁₀ is an alkyl group.

The term “amido” is art-recognized as an amino-substituted carbonyl and includes a moiety that can be represented by the general formula:

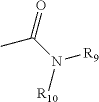

wherein R₉ and R₁₀ are as defined above.

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “aralkyl”, as used herein, refers to an alkyl group substituted with an aryl group (e.g., an aromatic or heteroaromatic group).

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, (C₁-C₁₀) alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, and —CN.

The term “carbonyl” is art-recognized and includes such moieties as can be represented by the general formula:

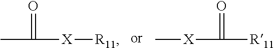

wherein X is a bond or represents an oxygen or a sulfur, and R₁₁ represents a hydrogen, an alkyl, a cycloalkyl, an alkenyl, an cycloalkenyl, or an alkynyl, R′₁₁ represents a hydrogen, an alkyl, a cycloalkyl, an alkenyl, an cycloalkenyl, or an alkynyl. Where X is an oxygen and R₁₁ or R′11 is not hydrogen, the formula represents an “ester”. Where X is an oxygen and R₁₁ is as defined above, the moiety is referred to herein as a carboxyl group, and particularly when R₁₁ is a hydrogen, the formula represents a “carboxylic acid”. Where X is an oxygen and R′₁₁ is hydrogen, the formula represents a “formate”. In general, where the oxygen atom of the above formula is replaced by sulfur, the formula represents a “thiocarbonyl” group. Where X is a sulfur and R₁₁ or R′11 is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “heteroatom” as used herein means an atom of any element other than carbon or hydrogen. Examples of heteroatoms are boron, nitrogen, oxygen, phosphorus, sulfur and selenium. Other heteroatoms include silicon and arsenic.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valencies of the heteroatoms.

In various aspects, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

The term “copolymer” as used herein, generally refers to a single polymeric material that is comprised of two or more different monomers. The copolymer can be of any form, such as random, block, graft, etc. The copolymers can have any end-group, including capped or acid end groups.

Vascular Grafts and Prostheses

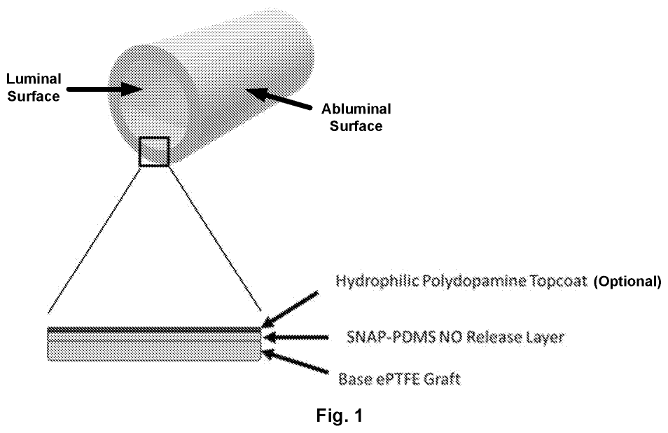

In various aspects, an implantable vascular graft is provided having a tubular base layer made from a graft material such as, but not limited to, ePTFE (expanded polytetraflouroethylene) or polyurethane, the tubular base layer defining a luminal surface and an abluminal surface; and a nitric oxide-releasing layer disposed on one or both of the luminal surface and the abluminal surface (see FIG. 1, in which the NO-releasing layer is disposed on the luminal surface of the tubular base layer). In some aspects, the nitric oxide-releasing layer is disposed on the luminal surface of the graft material. In some aspects, the nitric oxide-releasing layer is disposed on the abluminal surface of the graft material. The vascular grafts and prostheses described herein can be used to provide prolonged local delivery of nitric oxide when administered to a subject in need thereof. In some aspects, the vascular grafts and prostheses can include one or more additional therapeutic agents. The therapeutic agent can be an anti-proliferative agent such as sirolimus, paclitaxel, or a derivative thereof. In some aspects, the therapeutic agent can be incorporated within the tubular base layer, the nitric oxide-releasing layer, or both. In some aspects, the therapeutic agent can be coated onto a surface of the tubular base layer, a surface of the nitric oxide-releasing layer, or both.

In various aspects, the drug release from the graft material would be immediate after implantation and exposure to fluid (e.g. blood). Generally a “burst” effect of NO release would be expected, followed by sustained release for 30-120 days.

The implantable vascular grafts and prostheses include a graft material, e.g. a graft polymer. The graft material can include one or more thromboresistant (e.g. inhibiting blood clot formation or adhesion) materials, and in some aspects, the graft material is a thromboresistant polymer. In some aspects, the graft material is a thermoplastic elastomer. Suitable graft polymers can include a polyurethane, a polyethylene terephthalate, a polytetrafluoroethylene, a silicon, a copolymer thereof, or a blend thereof. In some aspects, the graft material is a polytetrafluoroethylene (PTFE). Biocompatible polymers can be formed as non-porous material or as a porous material with varying degrees and sizes of pores, as described below. Implantable medical devices can comprise one or both forms of biocompatible polymers, e.g. porous or non-porous material.

The thickness of the graft material may be selected to provide a desired loading of the therapeutic agent (if present), and desired mechanical properties of the graft, such as a suitable durability to the graft material or a desired minimum radius upon radial compression of the vascular graft after crimping. In some aspects, the graft material has a thickness of about 0.1 mm to about 0.8 mm, including intermediate ranges, such as, but not limited to, about 0.2 mm to about 0.6 mm, or about 0.3 mm to about 0.5 mm, etc.

The length of the vascular graft or prosthesis will typically be selected based on the intended application site. In some aspects, the vascular graft has a length of about 5 mm to about 500 mm, such as, but not limited to, about 5 mm to about 250 mm, about 10 mm to about 100 mm, about 20 mm to about 80 mm, about 20 mm to about 40 mm, about 40 mm to about 60 mm, or about 60 mm to about 80 mm.

The nitric oxide-releasing layer includes a polymer matrix, wherein the polymer matrix is made from a plurality of polysiloxanes and a plurality of nitric oxide-donating crosslinking moieties covalently crosslinking polysiloxanes in the plurality of polysiloxanes. In some aspects, each of the nitric oxide-donating crosslinking moieties in the plurality of nitric oxide-donating crosslinking moieties has a structure according to the following formula:

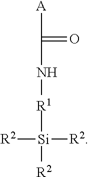

In the above formula, A is a nitric oxide donor; R¹ can be a substituted or unsubstituted alkyl, a substituted or unsubstituted heteroalkyl, a substituted or unsubstituted C₂-C₂₀ alkenyl, a substituted or unsubstituted C₂-C₂₀ herteroalkenyl, a substituted or unsubstituted C₂₀ alkoxy, or a substituted or unsubstituted C₁-C₂₀ heteroalkoxy; and each occurrence of R² is independently a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted alkoxy, a substituted or unsubstituted C₁-C₂₀ heteroalkoxy, or a bond to a polysiloxane in the plurality of polysiloxanes, provided that at least two occurrences of R² are a bond to a polysiloxane in the plurality of polysiloxanes. In some aspects, R¹ is a substituted or unsubstituted C₁-C₁₂ alkyl or a substituted or unsubstituted C₁-C₁₂ aminoalkyl. In some aspects, each occurrence of R² is a bond to a polysiloxane in the plurality of polysiloxanes.

The polymer matrix includes a plurality of polysiloxanes. In some aspects, the polysiloxanes in the plurality of polysiloxanes are selected from the group consisting of polydimethylsiloxane, polydiethylsiloxane, polydipropylsiloxane, and polydiphenylsiloxane. In some aspects, the plurality of polysiloxanes have a kinematic viscosity of about 2000 cSt to about 4000 cSt when not crosslinked in the polymer matrix.

In some aspects, the nitric oxide-donating crosslinking moieties are present in an amount from about 0.1 micromoles or greater (e.g., about 0.2 micromoles, about 0.3 micromoles, or about 0.35 micromoles or greater) per milligram of the polymer matrix. For example, the nitric oxide-donating crosslinking moieties can be present in an amount from about 0.1 micromoles to about 0.7 micromoles, about 0.2 micromoles to about 0.7 micromoles, about 0.3 micromoles to about 0.7 micromoles, about 0.35 micromoles to about 0.7 micromoles per milligram of the polymer matrix, or any intervening ranges, etc.

A variety of nitric oxide-donating groups can be employed in the nitric oxide-releasing layer of the vascular graft of the present disclosure. In some aspects, A in the structure above is an S-nitrosothiol. In some aspects, the S-nitrosothiol is selected from the group consisting of S-nitroso-N-acetyl-penicillamine, S-nitroso-N-acetyl cysteine, S-nitroso-N-acetyl cysteamine, S-nitrosoglutathione, methyl S-nitrosothioglycolate, and derivatives of any thereof. In some aspects, the nitric oxide donor is a diazeniumdiolate, e.g. diazeniumdiolated dibutylhexanediamine or a derivative thereof. In some aspects, A in the structure above has a structure according to the formula R⁴SNO, where R⁴ is an amino acid or fragment thereof.

In some aspects, the vascular graft further includes a coating layer disposed on the nitric oxide-releasing layer (FIG. 1). Suitable coating layers can include, for example, biocompatible and/or biodegradable polymers. In some aspects, the coating layer is a polydopamine. The coating layer can, in some aspects, provide for extended nitric oxide release. In some aspects, the coating layer can include a surface modifying agent such as 3,4-dihydroxyl-L-phenylalanine (DOPA), 3,4-dihydroxyphenylalanine methyl ester, dopamine, norepinephrine, or epinephrine.

In some aspects, nitric oxide release of coated grafts can be further modified by the addition of a hydrophilic coating (e.g. polydopamine and other various analogues of dopamine such as N-(3,4-dihydroxyphenethyl)acrylamide, N-(3,4-dihydroxyphenethyl)-2-mercaptoacetamide, and N-(3,4-dihydroxyphenethyl)pent-4-ynamide). The more hydrophilic the blood-polymer interface is, the higher the potential NO release to be emitted from the graft, which in turn can enhance the overall biocompatibility.

In some aspects, the vascular grafts and prostheses include a radially expandable support frame, wherein the graft material is attached to the radially expandable support frame. In some aspects, the radially expandable support frame is a self-expanding support frame. The support frame preferably supports the graft material in a desired configuration. The support frame can be formed from any suitable structure that maintains an attached graft material in a desired position, orientation or range of motion to perform a desired function. The specific implantable frame chosen will depend on several considerations, including the size and configuration of the vessel and the size and nature of the medical device. The frame can perform any desired function, including a stenting function. The frame configuration may be selected based on several factors, including the vessel in which the medical device is being implanted, the axial length of the treatment site, the inner diameter of the body vessel, and the desired delivery method for placing the support structure. Those skilled in the art can determine an appropriate stent based on these and other factors. The implantable frame can be sized so that the expanded configuration is slightly larger in diameter than the inner diameter of the vessel in which the medical device will be implanted. This sizing can facilitate anchoring of the medical device within the body vessel and maintenance of the medical device at a point of treatment following implantation.

The support frame may be formed from any suitable biocompatible material that allows for desired therapeutic effects upon implantation in a body vessel. Examples of suitable materials include, without limitation, any suitable metal or metal alloy, such as: stainless steels, nickel-titanium alloys including shape memory or superelastic types (e.g., nitinol or elastinite); inconel; noble metals including copper, silver, gold, platinum, palladium and iridium; refractory metals including molybdenum, tungsten, tantalum, titanium, rhenium, or niobium; stainless steels alloyed with noble and/or refractory metals; magnesium; amorphous metals; plastically deformable metals (e.g., tantalum); nickel-based alloys (e.g., including platinum, gold and/or tantalum alloys); iron-based alloys (e.g., including platinum, gold and/or tantalum alloys); cobalt-based alloys (e.g., including platinum, gold and/or tantalum alloys); cobalt-chrome; cobalt-chromium-nickel alloys; alloys of cobalt, nickel, chromium and molybdenum; cobalt-chromium-vanadium alloys; cobalt-chromium-tungsten alloys; platinum-iridium alloys; platinum-tungsten alloys; magnesium alloys; titanium alloys (e.g., TiC, TiN); tantalum alloys (e.g., TaC, TaN); bioabsorbable materials, including magnesium; or other biocompatible metals and/or alloys thereof.

In some embodiments, the implantable frames impart radially outward-directed force during deployment, whether self-expanding or radially-expandable. The radially outward-directed force can serve to hold the body lumen open against a force directed radially inward, as well as preventing restriction of the passageway through the lumen by intimal flaps or dissections generated by such as prior balloon angioplasty. Another function of the radially outward directed force can also fix the position of the stent within the body lumen by intimate contact between the stent and the walls of the lumen. In some aspects, the, the support frame is self-expanding. Upon compression, self-expanding frames can expand toward their pre-compression geometry. In some aspects, a self-expanding frame can be compressed into a low-profile delivery conformation and then constrained within a delivery system for delivery to a point of treatment in the lumen of a body vessel. Suitable implantable frames can also have a variety of configurations, including braided strands, helically wound strands, ring members, consecutively attached ring members, tube members, and frames cut from solid tubes. Also, suitable frames can have a variety of sizes. The exact configuration and size chosen will depend on several factors, including the desired delivery technique, the nature of the vessel in which the device will be implanted, and the size of the vessel. A frame structure and configuration can be chosen to facilitate maintenance of the device in the vessel following implantation. The implantable frame can be formed in any suitable shape, including a ring, a stent, a tube, or a zig-zag configuration. In some aspects, the implantable frame can be self-expanding or balloon-expandable.

In some aspects, the implantable frames can include one or more radiopaque markers.

Methods of Manufacture

Various methods are provided for making an implantable vascular graft or prosthesis described herein. The methods can include providing a vascular graft having a tubular base layer made from a graft material, the tubular base layer defining a luminal surface and an abluminal surface; and applying a polymer matrix to one or both of the luminal surface and the abluminal surface to form a nitric oxide-releasing layer. The polymer matrix forming the nitric oxide-releasing layer can be any of those described herein. The applying can include one or more of spraying, dip coating, casting, or otherwise depositing a solution of the polymer matrix and a suitable solvent (e.g. toluene, dichloromethane, or hexanes).

The methods can include making a tubular base layer made of a graft material, e.g. by (1) spraying, (2) dipping or (3) casting of the graft material in a solution, and drying the polymer around portions of a support frame. Alternatively, a dried sheet of graft material can be adhered to a support frame using an adhesive, sutures, UV-activated polymers, melting, or any suitable means of attachment providing a desirably durable attachment between the graft material and the frame. Preferably, a solution of the dissolved graft material can be coated onto a portion of the frame and attached to the frame as the solution is dried.

Solutions of the polymer matrix and/or the graft material can be prepared using a suitable solvent for the particular materials chosen. The solvent can be a volatile organic solvent such that the solution can be dried by removal of the organic solvent to form a portion of the implantable graft or prosthesis. In some aspects, the vascular graft further includes a coating layer disposed on the nitric oxide-releasing layer as described above. In some aspects, nitric oxide release of coated grafts can be further modified by the addition of a hydrophilic coating as described above.

For layers including a therapeutic agent, the therapeutic agent is preferably incorporated into the solution with the polymer and solvent. The concentration of the therapeutic agent in the solution can be adjusted depending upon the specific therapeutic agent and the application, e.g. about 10-500 mM, or about 50-300 mM in the organic solvent.

Methods of Use

The vascular grafts and prostheses described herein can be delivered to any suitable body vessel, including a vein, artery, biliary duct, ureteral vessel, body passage or portion of the alimentary canal. Methods for delivering vascular grafts and prostheses as described herein to any suitable body vessel are also provided, such as a vein, artery, biliary duct, ureteral vessel, body passage or portion of the alimentary canal. While many aspects discussed herein described the implantation of vascular grafts and prostheses in a vein, other aspects provide for implantation within other body vessels. In another matter of terminology there are many types of body canals, blood vessels, ducts, tubes and other body passages, and the term “vessel” is meant to include all such passages.

One method of deploying the vascular grafts and prostheses in a vessel involves radially compressing and loading the vascular grafts and prostheses into a delivery device, such as a catheter. A restraining means may maintain the vascular grafts and prostheses in the radially compressed configuration. For example, a self-expanding stent graft may be retained within a slidable sheath, while stent grafts that are not self-expanding may be crimped over a balloon portion of a delivery catheter. The compressed stent graft is thereby mounted on the distal tip of the delivery device, translated through a body vessel on the delivery device, and deployed from the distal end of the delivery device. For example, a delivery device may be a catheter having a pushing member adapted to urge the stent graft away from the delivery catheter. A sheath may be longitudinally translated relative to the stent graft to permit the stent graft to radially self-expand at the point of treatment within a body vessel. Alternatively, a balloon may be inflated to radially expand the stent graft.

Methods of treating a subject, which can be animal or human, are also provided. The methods can include the step of implanting one or more vascular grafts or prostheses as described herein. Methods of treatment can include the step of implanting one or more vascular grafts or prostheses configured to release a therapeutic agent, as described herein. In some embodiments, methods of treating may also include the step of delivering a vascular graft or prosthesis to a point of treatment in a body vessel, or deploying a vascular graft or prosthesis at the point of treatment.

Methods can include administering a vascular graft to an endoluminal surface of a vessel of a subject in need thereof, by providing a vascular graft described herein; intraluminally inserting the vascular graft and positioning the vascular graft at a location in the vessel expanding and anchoring the vascular graft at the location in the vessel of the subject. The vessel can include a vein, an artery, a biliary duct, a ureteral vessel, a body passage, or a portion of the alimentary canal.

In some aspects, the methods result in a subject having a decreased rate of infection following placement of the vascular graft as compared to a reference rate of infection for the otherwise same subject having the otherwise same vascular graft placed at the otherwise same location except where the vascular graft does not contain the nitric oxide-releasing layer.

In some aspects, the methods result in the vascular graft having an increased patency as compared to a reference patency for the otherwise same vascular graft except where the vascular graft does not contain the nitric oxide-releasing layer. The patency is measured at about the same period of time following administration in the otherwise same location of the otherwise same subject.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1: SNAP-PDMS Coated ePTFE Prosthetic AV Grafts

S-nitroso-N-acetylpenicillamine (SNAP), covalently attached to polydimethylsiloxane (PDMS), was used to coat ePTFE grafts in the form of a thin polymeric topcoat. In addition, polydopamine was utilized to control release rate. Polydopamine coatings were employed by submerging the SNAP-PDMS coated grafts in a solution of Tris buffer (pH 8.5) containing dopamine-HCl at a concentration of 2 mg mL⁻¹. ePTFE grafts with only SNAP-PDMS demonstrated a flux of 9.39×10⁻¹⁰ mol cm⁻² min⁻¹, while the grafts treated with polydopamine with 24 hr and 48 hr coating times had initial fluxes of 20.01×10⁻¹⁰ mol cm⁻² min and 27.85×10⁻¹⁰ mol cm⁻² min⁻¹, respectively.

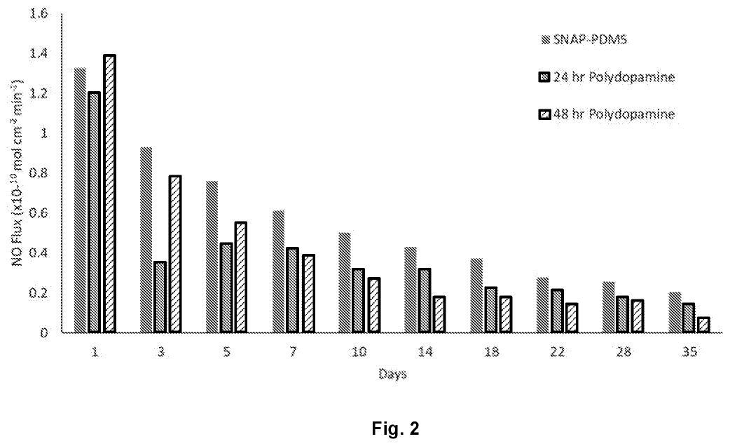

SNAP-PDMS coated 4 mm ePTFE grafts were placed in PBS at 37° C. for 35 days. FIG. 2 demonstrates sustained and measurable release of NO (as determined by NO flux) from the luminal ePTFE SNAP-PDMS coating for 35 days. The coated and uncoated grafts displayed initial NO release earlier, while the coated grafts demonstrated sustained release with their respective polydopamine coatings as shown in FIG. 2. Due to the increased hydrophilicity from the polydopamine layer, an increased NO flux is sustained, which over time exhausts the NO reservoir of the layer which leads to lower release rates over long periods of time.

### CLAUSES

Clause 1. An implantable vascular graft comprising: (1) a tubular base layer comprising a graft material, the tubular base defining a luminal surface and an abluminal surface; and (2) a nitric oxide-releasing layer disposed on one or both of the luminal surface and the abluminal surface; wherein the nitric oxide-releasing layer comprises a polymer matrix, wherein the polymer matrix comprises (i) a plurality of polysiloxanes; and (ii) a plurality of nitric oxide-donating crosslinking moieties covalently crosslinking polysiloxanes in the plurality of polysiloxanes; and wherein each of the nitric oxide-donating crosslinking moieties in the plurality of nitric oxide-donating crosslinking moieties have a structure according to the following formula:

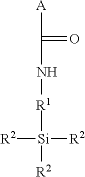

where A is a nitric oxide donor; where R¹ is a substituted or unsubstituted C₁-C₂₀ alkyl, a substituted or unsubstituted heteroalkyl, a substituted or unsubstituted C₂-C₂₀ alkenyl, a substituted or unsubstituted C₂-C₂₀ herteroalkenyl, a substituted or unsubstituted alkoxy, or a substituted or unsubstituted heteroalkoxy; where each occurrence of R² is independently a substituted or unsubstituted C₁-C₂₀ alkyl, a substituted or unsubstituted heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, a substituted or unsubstituted C₂₀ heteroalkoxy, or a bond to a polysiloxane in the plurality of polysiloxanes so long as at least two occurrences of R² are a bond to a polysiloxane in the plurality of polysiloxanes.

Clause 2. The implantable vascular graft according to clause 1, wherein the graft material is a thermoplastic elastomer.

Clause 3. The implantable vascular graft according to clause 1 or clause 2, wherein the graft material is selected from the group consisting of a polyurethane, a polyethylene terephthalate, a polytetrafluoroethylene, a silicon, a copolymer thereof, and a blend thereof.

Clause 4. The implantable vascular graft according to any one of clauses 1-3, wherein the graft material comprises a polytetrafluoroethylene (PTFE).

Clause 5. The implantable vascular graft according to any one of clauses 1-4, wherein the nitric oxide-releasing layer is disposed at least on the luminal surface of the graft material

Clause 6. The implantable vascular graft according to any one of clauses 1-5, wherein the nitric oxide-releasing layer is disposed at least on the abluminal surface of the graft material.

Clause 7. The implantable vascular graft according to any one of clauses 1-6, wherein A is an S-nitrosothiol.

Clause 8. The implantable vascular graft according to clause 7, wherein the S-nitrosothiol is selected from the group consisting of S-nitroso-N-acetyl-penicillamine, S-nitroso-N-acetyl cysteine, S-nitroso-N-acetyl cysteamine, S-nitrosoglutathione, methyl S-nitrosothioglycolate, and a derivative thereof.

Clause 9. The implantable vascular graft according to any one of clauses 1-6, wherein the nitric oxide donor is a diazeniumdiolate.

Clause 10. The implantable vascular graft according to clause 9, wherein the diazeniumdiolate is diazeniumdiolated dibutylhexanediamine or a derivative thereof.

Clause 11. The implantable vascular graft according to any one of clauses 1-6, wherein A has a structure according to the formula R⁴SNO, where R⁴ is an amino acid or fragment thereof.

Clause 12. The implantable vascular graft according to any one of clauses 1-11, wherein R¹ is a substituted or unsubstituted C₁-C₁₂ alkyl or a substituted or unsubstituted C₁-C₁₂ aminoalkyl.

Clause 13. The implantable vascular graft according to any one of clauses 1-12, wherein each occurrence of R² is a bond to a polysiloxane in the plurality of polysiloxanes.

Clause 14. The implantable vascular graft according to any one of clauses 1-13, wherein the polysiloxanes in the plurality of polysiloxanes are selected from the group consisting of polydimethylsiloxane, polydiethylsiloxane, polydipropylsiloxane, and polydiphenylsiloxane.

Clause 15. The implantable vascular graft according to any one of clauses 1-14, wherein the plurality of polysiloxanes have a kinematic viscosity of about 2000 cSt to about 4000 cSt when not crosslinked in the polymer matrix.

Clause 16. The implantable vascular graft according to any one of clauses 1-15, wherein the nitric oxide-donating crosslinking moieties are present in an amount from about 0.1 micromoles to 0.8 micromoles per milligram of the polymer matrix.

Clause 17. The implantable vascular graft according to any one of clauses 1-16, wherein the nitric oxide-donating crosslinking moieties are present in an amount from about 0.1 micromoles to about 0.7 micromoles, about 0.2 micromoles to about 0.7 micromoles, about 0.3 micromoles to about 0.7 micromoles, or about 0.35 micromoles to about 0.7 micromoles per milligram of the polymer matrix.

Clause 18. The implantable vascular graft according to any one of clauses 1-17, further comprising a coating layer disposed on the nitric oxide-releasing layer.

Clause 19. The implantable vascular graft according to clause 18, wherein the coating layer comprises a surface modifying agent selected from the group consisting of 3,4-dihydroxyl-L-phenylalanine (DOPA), 3,4-dihydroxyphenylalanine methyl ester, dopamine, norepinephrine, and epinephrine.

Clause 20. The implantable vascular graft according to any one of clauses 1-19, further comprising a radially expandable support frame, wherein the graft material is attached to the radially expandable support frame.

Clause 21. The implantable vascular graft according to clause 20, wherein the radially expandable support frame is a self-expanding support frame.

Clause 22. The implantable vascular graft according to any one of clauses 20-21, wherein the radially expandable support frame comprises a metal or metal alloy selected from the group consisting of a stainless steel, a nickel-titanium alloy, a noble metal, a refractory metal, a magnesium, an amorphous metal, a plastically deformable metal, a nickel-based alloy, an iron-based alloy, a cobalt-based alloy, a cobalt-chrome alloy, a cobalt-chromium-nickel alloy, a cobalt-chromium-vanadium alloy, a cobalt-chromium-tungsten alloy, a platinum-iridium alloy, a platinum-tungsten alloy, a magnesium alloy, a titanium alloy, a tantalum alloy, a bioabsorbable material, and a combination thereof.

Clause 23. The implantable vascular graft according to any one of clauses 1-22, wherein the vascular graft has a length of about 10 mm to about 100 mm, or about 20 mm to about 80 mm.

Clause 24. The implantable vascular graft according to any one of clauses 1-23, wherein the vascular graft has a fully expanded inner diameter of about 4 mm to about 25.

Clause 25. The implantable vascular graft according to any one of clauses 1-24, further comprising a therapeutic agent.

Clause 26. The implantable vascular graft according to clause 25, wherein the therapeutic agent is an anti-proliferative agent such as sirolimus, paclitaxel, or a derivative thereof.

Clause 27. The implantable vascular graft according to any one of clauses 25-26, wherein the therapeutic agent is incorporated within one or both of the tubular base layer and the nitric oxide-releasing layer.

Clause 28. The implantable vascular graft according to any one of clauses 25-27, wherein the therapeutic agent is coated onto a surface of one or both of the tubular base layer and the nitric oxide-releasing layer.

Clause 29. The implantable vascular graft according to any one of clauses 1-28, further comprising one or more anchoring means for anchoring the vascular graft to a surrounding blood vessel wall when the vascular graft is in an expanded state, wherein the anchoring means is selected from sutures and tissue glue.

Clause 30. The implantable vascular graft according to any one of clauses 1-29, further comprising one or more radiopaque markers.

Clause 31. A method of making an implantable vascular graft according to any one of claims 1-30, the method comprising: (1) providing a vascular graft comprising a tubular base layer comprising a graft material, the tubular base layer defining a luminal surface and an abluminal surface; and (2) applying a polymer matrix to one or both of the luminal surface and the abluminal surface to form a nitric oxide-releasing layer; wherein the polymer matrix comprises: (i) a plurality of polysiloxanes; and (ii) a plurality of nitric oxide-donating crosslinking moieties covalently crosslinking polysiloxanes in the plurality of polysiloxanes; and wherein each of the nitric oxide-donating crosslinking moieties in the plurality of nitric oxide-donating crosslinking moieties have a structure according to the following formula:

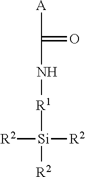

where A is a nitric oxide donor; where R¹ is a substituted or unsubstituted C₁-C₂₀ alkyl, a substituted or unsubstituted heteroalkyl, a substituted or unsubstituted C₂-C₂₀ alkenyl, a substituted or unsubstituted C₂-C₂₀ herteroalkenyl, a substituted or unsubstituted alkoxy, or a substituted or unsubstituted heteroalkoxy; where each occurrence of R² is independently a substituted or unsubstituted C₁-C₂₀ alkyl, a substituted or unsubstituted heteroalkyl, a substituted or unsubstituted C₂-C₂₀ alkenyl, a substituted or unsubstituted C₂-C₂₀ herteroalkenyl, a substituted or unsubstituted alkoxy, a substituted or unsubstituted C₂₀ heteroalkoxy, or a bond to a polysiloxane in the plurality of polysiloxanes so long as at least two occurrences of R² are a bond to a polysiloxane in the plurality of polysiloxanes.

Clause 32. The method according to clause 31, wherein the applying in step (2) comprises one or more of spraying, dip coating, casting, or otherwise depositing a solution comprising the polymer matrix and a suitable solvent.

Clause 33. The method according to clause 32, wherein the suitable solvent is selected from the group consisting of toluene, dichloromethane, and hexanes.

Clause 34. A method of administering a vascular graft to an endoluminal surface of a vessel of a subject in need thereof, the method comprising: intraluminally inserting a vascular graft according to any one of clauses 1-30 and positioning the vascular graft at a location in the vessel by means of a positioning apparatus; and expanding and anchoring the vascular graft at the location in the vessel of the subject.

Clause 35. The method according to clause 34, wherein the subject is a human.

Clause 36. The method according to clause 34 or clause 35, wherein the vessel is selected from the group consisting of a vein, an artery, a biliary duct, a ureteral vessel, a body passage, and a portion of the alimentary canal.

Clause 37. The method according to any one of clauses 34-36, wherein the subject has a decreased rate of infection following placement of the vascular graft as compared to a reference rate of infection for the otherwise same subject having the otherwise same vascular graft placed at the otherwise same location except where the vascular graft does not contain the nitric oxide-releasing layer.

Clause 38. The method according to any one of clauses 34-37, wherein the vascular graft has an increased patency as compared to a reference patency for the otherwise same vascular graft except where the vascular graft does not contain the nitric oxide-releasing layer, wherein the patency is measured at about the same period of time following administration in the otherwise same location of the otherwise same subject.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

We claim:

1. An implantable vascular graft comprising:
(1) a tubular base layer comprising a graft material, the tubular base layer defining a luminal surface and an abluminal surface; and
(2) a nitric oxide-releasing layer disposed on one or both of the luminal surface and the abluminal surface;
wherein the nitric oxide-releasing layer comprises a polymer matrix, wherein the polymer matrix comprises
(i) a plurality of polysiloxanes; and
(ii) a plurality of nitric oxide-donating crosslinking moieties covalently crosslinking polysiloxanes in the plurality of polysiloxanes; and
wherein each of the nitric oxide-donating crosslinking moieties in the plurality of nitric oxide-donating crosslinking moieties have a structure according to the following formula 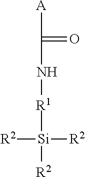
where A is a nitric oxide donor;
where R¹ is a substituted or unsubstituted C₁-C₂₀ alkyl, a substituted or unsubstituted C₂₀ heteroalkyl, a substituted or unsubstituted C₂-C₂₀ alkenyl, a substituted or unsubstituted C₂-C₂₀ herteroalkenyl, a substituted or unsubstituted alkoxy, or a substituted or unsubstituted heteroalkoxy; and
where each occurrence of R² is independently a substituted or unsubstituted alkyl, a substituted or unsubstituted heteroalkyl, a substituted or unsubstituted C₂-C₂₀ alkenyl, a substituted or unsubstituted C₂-C₂₀ herteroalkenyl, a substituted or unsubstituted C₂₀ alkoxy, a substituted or unsubstituted heteroalkoxy, or a bond to a polysiloxane in the plurality of polysiloxanes so long as at least two occurrences of R² are a bond to a polysiloxane in the plurality of polysiloxanes.

2. The implantable vascular graft according to claim 1, wherein the graft material is a thermoplastic elastomer.

3. The implantable vascular graft according to claim 2, wherein the graft material is selected from the group consisting of a polyurethane, a polyethylene terephthalate, a polytetrafluoroethylene, a silicon, a copolymer thereof, and a blend thereof.

4. The implantable vascular graft according to claim 1, wherein the graft material comprises a polytetrafluoroethylene (PTFE).

5. The implantable vascular graft according to claim 1, wherein the nitric oxide-releasing layer is disposed at least on the luminal surface of the graft material.

6. The implantable vascular graft according to claim 1, wherein the nitric oxide-releasing layer is disposed at least on the abluminal surface of the graft material.

7. The implantable vascular graft according to claim 1, wherein A is an S-nitrosothiol.

8. The implantable vascular graft according to claim 7, wherein the S-nitrosothiol is selected from the group consisting of S-nitroso-N-acetyl-penicillamine, S-nitroso-N-acetyl cysteine, S-nitroso-N-acetyl cysteamine, S-nitrosoglutathione, methyl S-nitrosothioglycolate, and derivatives of any thereof.

9. The implantable vascular graft according to claim 1, wherein the nitric oxide donor is a diazeniumdiolate.

10. The implantable vascular graft according to claim 9, wherein the diazeniumdiolate is diazeniumdiolated dibutylhexanediamine or a derivative thereof.

11. The implantable vascular graft according to claim 1, wherein A has a structure according to the formula R⁴SNO, where R⁴ is an amino acid or fragment thereof.

12. The implantable vascular graft according to claim 1, wherein R¹ is a substituted or unsubstituted C₁-C₁₂ alkyl or a substituted or unsubstituted C₁-C₁₂ aminoalkyl.

13. The implantable vascular graft according to claim 1, wherein each occurrence of R² is a bond to a polysiloxane in the plurality of polysiloxanes.

14. The implantable vascular graft according to claim 1, wherein the polysiloxanes in the plurality of polysiloxanes are selected from the group consisting of polydimethylsiloxane, polydiethylsiloxane, polydipropylsiloxane, polydiphenylsiloxane, and combinations thereof.

15. The implantable vascular graft according to claim 1, wherein the plurality of polysiloxanes have a kinematic viscosity of about 2000 cSt to about 4000 cSt when not crosslinked in the polymer matrix.

16. The implantable vascular graft according to claim 1, wherein the nitric oxide-donating crosslinking moieties are present in an amount from about 0.1 micromoles to 0.8 micromoles per milligram of the polymer matrix.

17. The implantable vascular graft according to claim 1, wherein the nitric oxide-donating crosslinking moieties are present in an amount from about 0.1 micromoles to about 0.7 micromoles, about 0.2 micromoles to about 0.7 micromoles, about 0.3 micromoles to about 0.7 micromoles, or about 0.35 micromoles to about 0.7 micromoles per milligram of the polymer matrix.

18. The implantable vascular graft according to claim 1, further comprising a coating layer disposed on the nitric oxide-releasing layer.

19. The implantable vascular graft according to claim 18, wherein the coating layer comprises a surface modifying agent selected from the group consisting of 3,4-dihydroxyl-L-phenylalanine (DOPA), 3,4-dihydroxyphenylalanine methyl ester, dopamine, norepinephrine, and epinephrine.

20. The implantable vascular graft according to any claim 1, further comprising a radially expandable support frame, wherein the graft material is attached to the radially expandable support frame.

21. The implantable vascular graft according to claim 20, wherein the radially expandable support frame is a self-expanding support frame.

22. The implantable vascular graft according to claim 20, wherein the radially expandable support frame comprises a metal or metal alloy selected from the group consisting of a stainless steel, a nickel-titanium alloy, a noble metal, a refractory metal, a magnesium, an amorphous metal, a plastically deformable metal, a nickel-based alloy, an iron-based alloy, a cobalt-based alloy, a cobalt-chrome alloy, a cobalt-chromium-nickel alloy, a cobalt-chromium-vanadium alloy, a cobalt-chromium-tungsten alloy, a platinum-iridium alloy, a platinum-tungsten alloy, a magnesium alloy, a titanium alloy, a tantalum alloy, a bioabsorbable material, and a combination thereof.

23. The implantable vascular graft according to claim 1, wherein the vascular graft has a length of about 10 mm to about 100 mm, or about 20 mm to about 80 mm.

24. The implantable vascular graft according to claim 1, wherein the vascular graft has a fully expanded inner diameter of about 4 mm to about 25.

25. The implantable vascular graft according to claim 1, further comprising a therapeutic agent.

26. The implantable vascular graft according to claim 25, wherein the therapeutic agent is an anti-proliferative agent selected from sirolimus, paclitaxel, or derivatives thereof.

27. The implantable vascular graft according to any one of claims 25-26, wherein the therapeutic agent is incorporated within one or both of the tubular base layer and the nitric oxide-releasing layer.

28. The implantable vascular graft according to any one of claims 25-26, wherein the therapeutic agent is coated onto a surface of one or both of the tubular base layer and the nitric oxide-releasing layer.

29. The implantable vascular graft according to claim 1, further comprising one or more anchoring means for anchoring the vascular graft to a surrounding blood vessel wall when the vascular graft is in an expanded state,

30. The implantable vascular graft according to claim 29, wherein the anchoring means is selected from sutures and tissue glue.

31. The implantable vascular graft according claim 1, further comprising one or more radiopaque markers.

32. A method of making an implantable vascular graft according to claim 1, the method comprising:
(1) providing a vascular graft comprising a tubular base layer comprising a graft material, the tubular base layer defining a luminal surface and an abluminal surface; and
(2) applying a polymer matrix to one or both of the luminal surface and the abluminal surface to form a nitric oxide-releasing layer;
wherein the polymer matrix comprises:
(i) a plurality of polysiloxanes; and
(ii) a plurality of nitric oxide-donating crosslinking moieties covalently crosslinking polysiloxanes in the plurality of polysiloxanes; and
wherein each of the nitric oxide-donating crosslinking moieties in the plurality of nitric oxide-donating crosslinking moieties have a structure according to the following formula 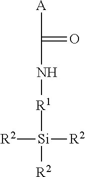
where A is a nitric oxide donor;
where R¹ is a substituted or unsubstituted C₁-C₂₀ alkyl, a substituted or unsubstituted C₂₀ heteroalkyl, a substituted or unsubstituted C₂-C₂₀ alkenyl, a substituted or unsubstituted C₂-C₂₀ herteroalkenyl, a substituted or unsubstituted alkoxy, or a substituted or unsubstituted heteroalkoxy; and
where each occurrence of R² is independently a substituted or unsubstituted alkyl, a substituted or unsubstituted heteroalkyl, a substituted or unsubstituted C₂-C₂₀ alkenyl, a substituted or unsubstituted C₂-C₂₀ herteroalkenyl, a substituted or unsubstituted C₂₀ alkoxy, a substituted or unsubstituted heteroalkoxy, or a bond to a polysiloxane in the plurality of polysiloxanes so long as at least two occurrences of R² are a bond to a polysiloxane in the plurality of polysiloxanes.

33. The method according to claim 32, wherein the applying in step (2) comprises one or more of spraying, dip coating, casting, or otherwise depositing a solution comprising the polymer matrix and a suitable solvent.

34. The method according to claim 33, wherein the suitable solvent is selected from the group consisting of toluene, dichloromethane, and hexanes.

35. A method of administering a vascular graft to an endoluminal surface of a vessel of a subject in need thereof, the method comprising:
intraluminally inserting a vascular graft according to claim 1 and positioning the vascular graft at a location in the vessel by use of a positioning apparatus; and
expanding and anchoring the vascular graft at the location in the vessel of the subject.

36. The method according to claim 35, wherein the subject is a human.

37. The method according to one of claim 35 or 36, wherein the vessel is selected from the group consisting of a vein, an artery, a biliary duct, a ureteral vessel, a body passage, and a portion of an alimentary canal.

38. The method according to any one of claims 35-36, wherein the subject has a decreased rate of infection following placement of the vascular graft as compared to a reference rate of infection for the otherwise same subject having the otherwise same vascular graft placed at the otherwise same location except where the vascular graft does not contain the nitric oxide-releasing layer.

39. The method according to any one of claims 35-36, wherein the vascular graft has an increased patency as compared to a reference patency for the otherwise same vascular graft except where the vascular graft does not contain the nitric oxide-releasing layer, wherein the patency is measured at about the same period of time following administration in the otherwise same location of the otherwise same subject.
